(19) United States
(12) Patent Application Publication
(10) Pub. No.: US 2023/0330057 A1
(43) Pub. Date: Oct. 19, 2023

(54) COMPOSITIONS AND METHODS FOR PROMOTING SLEEP

(71) Applicant: ALL THINGS HARVESTED LLC, San Juan Capistrano, CA (US)

(72) Inventor: Jacob Chavez, San Juan Capistrano, CA (US)

(21) Appl. No.: 17/721,789

(22) Filed: Apr. 15, 2022

(51) Int. Cl. A61K 31/352; A61K 31/05; A61K 31/197; A61K 31/4045; A61K 9/00

(52) U.S. Cl. CPC A61K 31/352; A61K 31/05; A61K 31/197; A61K 31/4045; A61K 9/0053

(57) ABSTRACT

A composition for promoting sleep includes a mixture of cannabinoids and a carrier. The mixture of cannabinoids includes cannabidiol (CBD), cannabigerol (CBG), and cannabinol (CBN). The composition may further include one or more supplemental active ingredients. The one or more supplemental active ingredients may include L-theanine, GABA, 5-HTP and/or melatonin.

### BACKGROUND

Poor sleep, or not enough sleep, is a common problem faced by many people, and can lead to physical or physiological distress. For example, poor sleep can negatively impact hormones, exercise performance and brain function, and can lead to weight gain and increased risk of disease. While many prescription, and even over-the-counter, sleep aids exist, such medications can have unwanted side-effects. Many people therefore turn to natural, or herbal, sleep aid supplements. However, such natural supplements have mixed results—some work for some people, others do not. Accordingly, more effective, natural sleep aids are desirable.

### SUMMARY

According to embodiments of the present disclosure, a composition for promoting sleep includes a mixture of cannabinoids and a carrier. The mixture of cannabinoids may include cannabidiol (CBD), cannabigerol (CBG), and cannabinol (CBN). In some embodiments, the CBD may be present in the mixture of cannabinoids in an amount of about 50 wt % to about 90 wt % based on a total weight of the mixture of cannabinoids. The CBG may be present in the mixture of cannabinoids in an amount of about 5 wt to about 30 wt % based on a total weight of the mixture of cannabinoids. And the CBN may be present in the mixture of cannabinoids in an amount of about 1 wt % to about 30 wt % based on a total weight of the mixture of cannabinoids.

In some embodiments, a weight ratio of the CBG to the CBD in the mixture of cannabinoids may be about 1:20 to about 1:1. A weight ratio of the CBN to the CBD in the mixture of cannabinoids may be about 1:50 to about 1:1. And a weight ratio of the CBN to about CBG in the mixture of cannabinoids may be about 1:5 to about 1:1.

According to some embodiments, a weight ratio of the CBN to the CBD may be about 1:50 to about 1:1, or a weight ratio of the CBN to the CBG may be about 1:5 to about 1:1.

In some embodiments, the carrier may include a carrier oil or a carrier matrix. The carrier oil may include a tree-based oil, a nut-based oil or a plant-based oil. The carrier oil may include a medium-chain triglyceride (MCT) oil, an olive oil, an avocado oil, an almond oil, or a hemp seed oil.

According to some embodiments, the carrier matrix may include a gummy composition. The gummy composition may include a gelatin-based gummy base composition or a plant-based gummy base composition.

In some embodiments, the composition may further include one or more supplemental active ingredients. The one or more supplemental active ingredients may include one or more of L-theanine, 5-hydroxytryptophan (5-HTP), gamma aminobutyricacid (GABA), and melatonin.

According to some embodiments, a weight ratio of the mixture of cannabinoids to a sum total of the supplemental active ingredients may be about 1:5 to about 1:2. A sum total amount of the supplemental active ingredients in the composition may be about 0.1 wt % to about 1 wt % based on a total amount of the mixture of cannabinoids, the supplemental active ingredients, and the carrier.

In some embodiments, the one or more supplemental active ingredients may include each of L-theanine, GABA, 5-HTP and melatonin. The L-theanine may be present in an amount of about 20 wt % to about 100 wt % based on a total weight of the supplemental active ingredients. The 5-HTP may be present in an amount of about 20 wt % to about 100 wt % based on a total weight of the supplemental active ingredients. The GABA may be present in an amount of about 5 wt % to about 100 wt % based on a total weight of the supplemental active ingredients. The melatonin may be present in an amount of about 5 wt % to about 100 wt % based on a total weight of the supplemental active ingredients.

In some embodiments, a weight ratio of the L-theanine to the GABA in the supplemental active materials may be about 1:0.1 to about 1:0.5. A weight ratio of the L-theanine to the 5-HTP in the supplemental active materials may be about 1:0.5 to about 1:2. A weight ratio of the L-theanine to the melatonin in the supplemental active materials may be about 1:0.1 to about 1:0.5. A weight ratio of the 5-HTP to the GABA in the supplemental active materials may be about 1:0.1 to about 1:0.5. A weight ratio of the 5-HTP to the melatonin in the supplemental active materials may be about 1:0.1 to about 1:0.5. A weight ratio of the GABA to the melatonin in the supplemental active materials may be about 1:0.5 to about 1:2.

According to some embodiments, a method of promoting sleep includes administering to a user the compositions described herein prior to a predetermined sleep time. In some embodiments, administering the composition comprises orally administering the composition.

### DETAILED DESCRIPTION

According to embodiments of the present disclosure, compositions for promoting sleep include a mixture of different non-psychoactive cannabinoids in a carrier. In some embodiments, for example, the mixture of different cannabinoids may include at least two different cannabinoids. And in some embodiments, the mixture of different cannabinoids includes at least three different cannabinoids, or three different cannabinoids. For example, in some embodiments, the mixture of cannabinoids may include cannabidiol (CBD), cannabigerol (CBG), and cannabinol (CBN).

CBD, CBG and CBN are all non-psychoactive cannabinoids that serve different purposes in the mixture of cannabinoids, and work synergistically to help promote sleep. For example, CBD may promote relaxation and relieve anxiety and stress, which are some of the major causes of poor sleep. CBG, on the other hand, may promote recovery and provide pain relief. And CBN may promote relaxation and provide some anti-inflammatory effect. Taken together, these three cannabinoids may help users to fall asleep faster, stay asleep longer, and experience more restful, restorative sleep.

In some embodiments, the CBD may be present in the mixture of cannabinoids in a weight range of about 50 wt % to about 90 wt %, for example about 55 wt % to about 90 wt %, about 60 wt % to about 90 wt %, about 65 wt % to about 90 wt %, about 70 wt % to about 90 wt %, about 50 wt % to about 85 wt %, about 50 wt % to about 80 wt %, about 50 wt % to about 75 wt %, about 55 wt % to about 85 wt %, about 55 wt % to about 80 wt %, about 55 wt % to about 75 wt %, about 60 wt % to about 85 wt %, about 60 wt % to about 75 wt %, about 65 wt % to about 85 wt %, about 65 wt % to 75 wt %, about 70 wt % to about 85 wt %, about 70 wt % to about 80 wt %, or about 70 wt % to about 75 wt %, based on the total, combined weight of the cannabinoids in the mixture (e.g., the total, combined weight of CBD, CBG and CBN).

In some embodiments, the CBG may be present in the mixture of cannabinoids in a weight range of about 5 wt % to about 30 wt %, for example about 6 wt % to about 30 wt %, about 7 wt % to about 30 wt %, about 8 wt % to about 30 wt %, about 9 wt % to about 30 wt %, about 10 wt % to about 30 wt %, about 5 wt % to about 25 wt %, about 5 wt % to about 20 wt %, about 5 wt % to about 15 wt %, about 10 wt % to about 25 wt %, about 10 wt % to about 20 wt %, or about 10 wt % to about 15 wt %, based on the total, combined weight of the cannabinoids in the mixture (e.g., the total, combined weight of CBD, CBG and CBN).

In some embodiments, the CBN may be present in the mixture of cannabinoids in a weight range of about 1 wt % to about 30 wt %, for example about 2 wt % to about 30 wt %, about 3 wt % to about 30 wt %, about 4 wt % to about 30 wt %, about 5 wt % to about 30 wt %, about 10 wt % to about 30 wt %, about 1 wt % to about 25 wt %, about 1 wt % to about 20 wt %, about 1 wt % to about 15 wt %, about 2 wt % to about 25 wt %, about 2 wt % to about 20 wt %, or about 2 wt % to about 15 wt %, based on the total, combined weight of the cannabinoids in the mixture (e.g., the total, combined weight of CBD, CBG and CBN).

According to some embodiments, a weight ratio of CBG to CBD in the mixture of cannabinoids may be about 1:20 to about 1:1, for example about 1:20 to about 1:2, about 1:20 to about 1:3, about 1:20 to about 1:4, about 1:20 to about 1:5, 1:10 to about 1:1, about 1:10 to about 1:2, about 1:10 to about 1:3, about 1:10 to about 1:4, about 1:10 to about 1:5, or about 1:5, 1:10, or about 1:2.

In some embodiments, a weight ratio of the CBN to CBD in the mixture of cannabinoids may be about 1:50 to about 1:1, for example about 1:50 to about 1:2, about 1:50 to about 1:3, about 1:50 to about 1:4, about 1:50 to about 1:5, 1:40 to about 1:1, about 1:40 to about 1:2, about 1:40 to about 1:3, about 1:40 to about 1:4, about 1:40 to about 1:5, or about 1:40, 1:5 or about 1:3. And in some embodiments, the weight ratio of CBN to CBG in the mixture of cannabinoids may be about 1:5 to about 1:1, for example about 1:4 to about 1:1, about 1:3 to about 1:1, or about 1:2 to about 1:1, or about 1:4, 1:1 or about 1:1.5.

The mixture of cannabinoids (e.g., the total amount of CBG, CBD and CBN) may be present in the composition in an amount of about 0.1 wt % to about 10 wt %, for example about 0.2 wt % to about 10 wt %, 0.3 wt % to about 10 wt %, 0.4 wt % to about 10 wt %, 0.5 wt % to about 10 wt %, 0.1 wt % to about 9 wt %, 0.1 wt % to about 8 wt %, 0.1 wt % to about 7 wt %, 0.2 wt % to about 9 wt %, 0.2 wt % to about 8 wt %, 0.2 wt % to about 7 wt %, 0.3 wt % to about 9 wt %, 0.3 wt % to about 8 wt %, 0.3 wt % to about 7 wt %, 0.4 wt % to about 9 wt %, 0.4 wt % to about 8 wt %, 0.4 wt % to about 7 wt %, 0.5 wt % to about 9 wt %, 0.5 wt % to about 8 wt %, 0.5 wt % to about 7 wt %, or about 0.5 wt % or about 6.5 wt %.

In the composition, the carrier is not particularly limited, and may be any carrier suitable to suspend, disperse or dissolve the mixture of different cannabinoids. In some embodiments, for example, the carrier may include a carrier oil or other carrier solvent suitable to dissolve, disperse or suspend the mixture of cannabinoids. For example, in some embodiments, the carrier may include a tree-, nut- or plant-based oil, non-limiting examples of which include medium-chain triglyceride (MCT) oils, olive oils, avocado oils, almond oils, hemp seed oils, etc. In some embodiments, for example, the carrier may include an MCT oil. The MCT oil is also not particularly limited, and may be any suitable MCT oil, non-limiting examples of which include those derived from coconut oils or palm kernel oils.

In some embodiments, the carrier may include a carrier matrix in which the mixture of cannabinoids is entrapped, encased, or otherwise suspended or dispersed. The carrier matrix is not particularly limited, and may be any matrix material suitable for the intended application of the composition. For example, in some embodiments, the carrier matrix may be such that the composition is a gel having a viscosity sufficient to enable rubbing or other topical application to the skin. Non-limiting examples of suitable such matrix materials include polylactide solutions with high molecular weight poly(ethylene oxide), highly dispersed silica, high-melting point fats or triglycerides (e.g., hardened glycerides of saturated fatty acids having a chain length of, for example, C14-C24 and a melting point above 50° C. to 90° C., which can be either vegetable- or plant-based or animal-based), a mixture of alcohol (e.g., isopropyl alcohol) and a fatty acid, soluble collagen, wheat germ oil, castor oil, soy bean oil, and glycerol oil.

In other embodiments, however, the matrix may be such that the mixture of cannabinoids is entrapped, encased, or otherwise suspended or dispersed in a more solid matrix. For example, in some embodiments, the matrix material may be a material suitable to entrap, encase or otherwise suspend the mixture of cannabinoids in an edible “gummy” composition. The carrier matrix materials for preparing such a gummy composition are not particularly limited, and may be any suitable, food-grade matrix materials capable of forming a gummy composition. For example, in some embodiments, the gummy composition may be gelatin-based or plant-based (e.g., pectin-based). However, as demand for plant-based dietary supplements is growing, in some embodiments, the gummy composition may be pectin-based. For example, in some embodiments, a pectin-based gummy composition may include the mixture of cannabinoids (as a suspended or entrapped active ingredient complex) encased, entrapped or otherwise suspended in a gummy base composition. In some embodiments, the gummy base composition may include gelling component (e.g., pectin or gelatin) and a solvent capable of hydrating the gelling component (e.g., pectin or gelatin). The solvent is not particularly limited, and may be any suitable, food-grade solvent. In some embodiments, for example, the solvent may be or include water. Methods of making gummy compositions are known to those of ordinary skill in the art, and are therefore not described in detail here.

In some embodiments, the gummy base composition may further include certain additives. The additives are not particularly limited, and may include any food-grade additives conventionally used in gummy compositions. For example, in some embodiments, the additives may include one or more of flavoring agents, sweeteners, gelation/coagulation promoters, buffers, starches, colorants, preservatives, etc. The flavoring agents and sweeteners are not particularly limited, and may include any suitable food-grade flavorants and sweeteners, some non-limiting examples of which include flavored liquids or syrups, sugars, and sugar-free sweeteners (e.g., erythritol, sucralose, stevia, aspartame, etc.). Similarly, the colorants are also not particularly limited, and may be any suitable food-grade colorant.

The gelation/coagulation promoter is also not particularly limited, and may be any suitable such component, which may differ based on the gelling component in the composition. Some non-limiting examples of suitable gelation/coagulation promoters include food-grade acids, e.g., citric acid, malic acid, lactic acid, fumaric acid, etc. In some embodiments, for example, the gelation/coagulation promoter may include citric acid.

The buffer is also not particularly limited, and may be any suitable food-grade buffer, non-limiting examples of which include salts of food-grade acids, e.g., sodium citrate, and the like.

The starch serves to help thicken and gel the gummy base composition, and is also not particularly limited. For example, in some embodiments, the starch may be any plant-based starch source, some non-limiting examples of which include corn starch, potato starch, wheat starch, tapioca starch, etc. In some embodiments, for example the starch may include tapioca starch. Additionally, the starch may be present in the gummy base composition as a liquid or syrup (e.g., a corn syrup, or a tapioca syrup).

In some embodiments, for example, the gummy base composition may include the gelling components (e.g., pectin or gelatin) and water (or other suitable solvent) in addition to a gelation/coagulation promoter, a buffer and a starch. And in some embodiments, the gummy base composition may further include one or more of a flavorant, a colorant, a preservative, and/or a sweetener. In some example embodiments, the gummy composition may include pectin (as a gelling component), water, a tapioca syrup (as a starch), a sweetener, citric acid (as a gelation/coagulation promoter), sodium citrate (as a buffer), a flavorant, and a colorant.

According to some embodiments, the gummy base composition may include the gelling component (e.g., pectin) and the buffer (e.g., sodium citrate) in a combined amount of about 5 wt % to about 10 wt % based on a total weight of the gummy composition (e.g., a total combined weight of the gelling component, solvent, gelation/coagulation promoter, buffer, starch, sweetener, flavorants and colorants). For example, in some embodiments, the gelling component (e.g., pectin) and the buffer (e.g., sodium citrate) may be present in a combined amount of about 6 wt % to about 8 wt % based on a total weight of the gummy composition. Additionally, in some embodiments, the gelling component (e.g., pectin) and the buffer (e.g., sodium citrate) may be present in the overall composition (including the mixture of cannabinoids and optional supplemental active ingredients) in a combined amount of about 5 wt % to about 10 wt % based on a total weight of the overall composition (including the mixture of cannabinoids and optional supplemental active ingredients). For example, in some embodiments, the gelling component (e.g., pectin) and the buffer (e.g., sodium citrate) may be present in a combined amount of about 6 wt % to about 8 wt % based on a total weight of the overall composition.

And in some embodiments, the gelation/coagulation promoter (e.g., citric acid) may be present in an amount of about 0.1 wt % to about 0.5 wt % based on a total weight of the gummy composition (e.g., a total combined weight of the gelling component, solvent, gelation/coagulation promoter, buffer, starch, sweetener, flavorants and colorants). For example, in some embodiments, the gelation/coagulation promoter (e.g., citric acid) may be present in an amount of about 0.1 wt % to about 0.3 wt % based on a total weight of the gummy composition. Additionally, in some embodiments, the gelation/coagulation promoter may be present in the overall composition (including the mixture of cannabinoids and optional supplemental active ingredients) in an amount of about 0.1 wt % to about 0.5 wt % based on a total weight of the overall composition (including the mixture of cannabinoids and optional supplemental active ingredients). For example, in some embodiments, the gelation/coagulation promoter may be present in an amount of about 0.1 wt % to about 0.3 wt % based on a total weight of the overall composition.

According to some embodiments, the gummy base composition may include the starch (e.g., tapioca syrup) in an amount of about 15 wt % to about 50 wt % based on a total weight of the gummy composition (e.g., a total combined weight of the gelling component, solvent, gelation/coagulation promoter, buffer, starch, sweetener, flavorants and colorants). For example, in some embodiments, the starch (e.g., tapioca syrup) may be present in an amount of about 20 wt % to about 40 wt % based on a total weight of the gummy composition. Additionally, in some embodiments, the starch may be present in the overall composition (including the mixture of cannabinoids and optional supplemental active ingredients) in an amount of about 15 wt % to about 50 wt % based on a total weight of the overall composition (including the mixture of cannabinoids and optional supplemental active ingredients). For example, in some embodiments, the starch may be present in an amount of about 20 wt % to about 40 wt % based on a total weight of the overall composition.

In some embodiments, the sweetener (e.g., erythritol) may be present in an amount of about 25 wt % to about 60 wt % based on a total weight of the gummy composition (e.g., a total combined weight of the gelling component, solvent, gelation/coagulation promoter, buffer, starch, sweetener, flavorants and colorants). For example, in some embodiments, the sweetener (e.g., erythritol) may be present in an amount of about 30 wt % to about 50 wt % based on a total weight of the gummy composition. Additionally, in some embodiments, the sweetener may be present in the overall composition (including the mixture of cannabinoids and optional supplemental active ingredients) in an amount of about 25 wt % to about 60 wt % based on a total weight of the overall composition (including the mixture of cannabinoids and optional supplemental active ingredients). For example, in some embodiments, the sweetener may be present in an amount of about 30 wt % to about 50 wt % based on a total weight of the overall composition.

According to some embodiments, the gummy base composition may include the colorant(s) in an amount of greater than 0 wt % to about 1 wt % based on a total weight of the gummy composition (e.g., a total combined weight of the gelling component, solvent, gelation/coagulation promoter, buffer, starch, sweetener, flavorants and colorants). For example, in some embodiments, the colorant(s) may be present in an amount of greater than 0 wt % to about 0.7 wt %, or about 0.01 wt % to about 0.5 wt % based on a total weight of the gummy composition. Additionally, in some embodiments, the colorant(s) may be present in the overall composition (including the mixture of cannabinoids and optional supplemental active ingredients) in an amount of greater than 0 wt % to about 1 wt % based on a total weight of the overall composition (including the mixture of cannabinoids and optional supplemental active ingredients). For example, in some embodiments, the colorant(s) may be present in an amount of greater than 0 wt % to about 0.7 wt %, or about 0.01 wt % to about 0.5 wt % based on a total weight of the overall composition. However, it is understood the colorant(s) may also be omitted.

In some embodiments, the gummy base composition may include the flavorant(s) in an amount of greater than 0 wt % to about 5 wt % based on a total weight of the gummy composition (e.g., a total combined weight of the gelling component, solvent, gelation/coagulation promoter, buffer, starch, sweetener, flavorants and colorants). For example, in some embodiments, the flavorant(s) may be present in an amount of about 1 wt % to about 4 wt %, or about 1 wt % to about 3 wt % based on a total weight of the gummy composition. Additionally, in some embodiments, the flavorant(s) may be present in the overall composition (including the mixture of cannabinoids and optional supplemental active ingredients) in an amount of greater than 0 wt % to about 5 wt % based on a total weight of the overall composition (including the mixture of cannabinoids and optional supplemental active ingredients). For example, in some embodiments, the colorant(s) may be present in an amount of about 1 wt % to about 4 wt %, or about 1 wt % to about 3 wt % based on a total weight of the overall composition. However, it is understood the colorant(s) may also be omitted. However, it is understood the flavorant(s) may also be omitted.

And according to some embodiments, the solvent (e.g., water) may be present in an amount of about 10 wt % to about 30 wt % based on a total weight of the gummy composition (e.g., a total combined weight of the gelling component, solvent, gelation/coagulation promoter, buffer, starch, sweetener, flavorants and colorants). For example, in some embodiments, the solvent (e.g., water) may be present in an amount of about 15 wt % to about 25 wt % based on a total weight of the gummy composition. Additionally, in some embodiments, the solvent may be present in the overall composition (including the mixture of cannabinoids and optional supplemental active ingredients) in an amount of about 10 wt % to about 30 wt % based on a total weight of the overall composition (including the mixture of cannabinoids and optional supplemental active ingredients). For example, in some embodiments, the solvent may be present in an amount of about 15 wt % to about 25 wt % based on a total weight of the overall composition. However, it is understood the colorant(s) may also be omitted.

In some embodiments, for example, when the carrier is a carrier oil, the composition may include a tincture for promoting sleep. The tincture may be used topically on the skin, or may be ingested orally in order to impart the sleep promotion effect. According to some embodiments, the sleep promotion tincture may include the mixture of cannabinoids, as described above, and the carrier may include a carrier oil, for example, an MCT oil. In some embodiments, when used as a tincture for promoting sleep, the weight ratio of the mixture of cannabinoids to the carrier oil may be about 1:20, for example about 1:19, about 1:17, about 1:16, about 1:15, about 1:14 or about 1:13. And in some embodiments, a recommended dosage per application or administration (e.g., oral administration) may be about 1 to 2 mL tincture for oral administration, and about 3 mL to about 8 mL for topical application.

In some embodiments, for example, when the carrier is a carrier matrix, the composition may include an edible gummy for promoting sleep. The edible gummy may include the mixture of cannabinoids encased, entrained, entrapped, or otherwise suspended or dispersed in the carrier matrix (e.g., the gelled gummy base composition). In some embodiments, when used as a gummy for promoting sleep, the mixture of cannabinoids may be present in the gummy in an amount of about 0.01 wt % to about 1 wt %, based on a total weight percent of the gelled gummy, for example, about 0.01 wt % to about 0.9 wt %, 0.01 wt % to about 0.8 wt %, 0.01 wt % to about 0.7 wt %, 0.01 wt % to about 0.6 wt %, 0.01 wt % to about 0.5 wt %, 0.01 wt % to about 0.4 wt %, 0.01 to about 0.3 wt %, 0.1 wt % to about 1 wt %, about 0.1 wt % to about 0.9 wt %, 0.1 wt % to about 0.8 wt %, 0.1 to about 0.7 wt %, 0.1 wt % to about 0.6 wt %, 0.1 wt % to about 0.5 wt %, 0.1 wt % to about 0.4 wt %, about 0.1 to about 0.3 wt %, or about 0.1 wt % to about 0.25 wt %. And in some embodiments, a recommended dosage per administration may be 1 to 2 gummies per administration (e.g., 1 to 2 gummies prior to dedicated sleep time). In some embodiments, a total weight of each gummy may be about 3 grams to about 10 grams, for example, about 3 grams to about 9 grams, about 3 grams to about 8 grams, about 3 grams to about 7 grams, about 3 grams to about 6 grams, or about 6 grams.

According to some embodiments, in addition to the mixture of cannabinoids and the carrier, the compositions for promoting sleep may further include one or more supplemental active ingredients. In some embodiments, for example, the one or more supplemental active ingredients may include L-theanine, GABA (gamma aminobutyric acid), 5-HTP (5-hydroxytryptophan), and/or melatonin. L-theanine is an amino acid found primarily in green and black tea and some mushrooms, and some recent studies link L-theanine with easing anxiety and stress, and reducing insomnia. GABA is a naturally occurring amino acid that works as a neurotransmitter in the brain, and may have a calming and relaxing effect on the nervous system, thereby helping to improve sleep. 5-HTP increases the production of serotonin, which may lessen the symptoms associated with depression and other mood disorders, ease pain, and improve sleep. Melatonin is a natural hormone in the brain whose production and release is connected to the time of day (e.g., increasing when dark, and decreasing when light), and has been associated with the treatment of insomnia and jet lag. When one or more of these supplemental active ingredients combine with the mixture of cannabinoids, they may interact synergistically to provide an increased sleep promotion/improvement effect. In some embodiment, the compositions for sleep promotion include the mixture of cannabinoids and carrier discussed above in addition to each of L-theanine, 5-HTP, GABA and melatonin.

In some embodiments, a weight ratio of the mixture of cannabinoids to the sum total of the supplemental active ingredients may be about 1:5 to about 1:2, for example about 1:4 to about 1:2, about 1:3 to about 1:2, or about 1:2, about 1:8, or about 1:1.75.

According to some embodiments, a sum total amount of the supplemental active ingredients in the composition may be about 0.1 wt % to about 1 wt % based on a total amount of the mixture of cannabinoids, supplemental active ingredients, and the carrier. For example, in some embodiments, the sum total amount of the supplemental active ingredients in the composition may be about 0.1 wt % to about 0.9 wt %, about 0.1 wt % to about 0.8 wt %, 0.1 wt % to about 0.7 wt %, about 0.1 wt % to about 0.6 wt %, about 0.1 wt % to about 0.5 wt %, about 0.1 to about 0.4 wt %, or about 0.4 wt %.

According to some embodiments, the L-theanine may be present in the composition in an amount of about 20 wt % to about 100 wt % based on a total weight of all of the supplemental active ingredients. For example, in some embodiments, the L-theanine may be present in an amount of about 20 wt % to about 90 wt %, about 20 wt % to about 80 wt %, about 20 wt % to about 70 wt %, about 20 wt % to about 60 wt %, about 20 wt % to about 50 wt %, about 30 wt % to about 90 wt %, about 30 wt % to about 80 wt %, about 30 wt % to about 70 wt %, about 30 wt % to about 60 wt %, about 30 wt % to about 50 wt %, or about 35 wt % to about 45 wt % based on a total weight of all of the supplemental active ingredients.

Similarly, in some embodiments, the 5-HTP may be present in the composition in an amount of about 20 wt % to about 100 wt % based on a total weight of all of the supplemental active ingredients. For example, in some embodiments, the 5-HTP may be present in an amount of about 20 wt % to about 90 wt %, about 20 wt % to about 80 wt %, about 20 wt % to about 70 wt %, about 20 wt % to about 60 wt %, about 20 wt % to about 50 wt %, about 30 wt % to about 90 wt %, about 30 wt % to about 80 wt %, about 30 wt % to about 70 wt %, about 30 wt % to about 60 wt %, about 30 wt % to about 50 wt %, or about 35 wt % to about 45 wt % based on a total weight of all of the supplemental active ingredients.

Additionally, in some embodiments, the GABA may be present in the composition in an amount of about 5 wt % to about 100 wt % based on a total weight of all of the supplemental active ingredients. For example, in some embodiments, the GABA may be present in an amount of about 5 wt % to about 50 wt %, about 5 wt % to about 40 wt %, about 5 wt % to about 30 wt %, about 5 wt % to about 20 wt %, or about 5 wt % to about 10 wt %, based on a total weight of all of the supplemental active ingredients.

Similarly, in some embodiments, the melatonin may be present in the composition in an amount of about 5 wt % to about 100 wt % based on a total weight of all of the supplemental active ingredients. For example, in some embodiments, the melatonin may be present in an amount of about 5 wt % to about 50 wt %, about 5 wt % to about 40 wt %, about 5 wt % to about 30 wt %, about 5 wt % to about 20 wt %, or about 5 wt % to about 10 wt %, based on a total weight of all of the supplemental active ingredients.

Also, in some embodiments, a weight ratio of L-theanine to GABA in the supplemental active ingredients may be about 1:0.1 to about 1:0.5, for example, about 1:0.1 to about 1:0.4, about 1:0.1 to about 1:0.3, or about 1:0.2. Similarly, a weight ratio of L-theanine to melatonin in the supplemental active ingredients may be about 1:0.1 to about 1:0.5, for example, about 1:0.1 to about 1:0.4, about 1:0.1 to about 1:0.3, or about 1:0.2.

In some embodiments, a weight ratio of L-theanine to 5-HTP in the supplemental active ingredients may be about 1:0.5 to about 1:2, for example, about 1:0.5 to about 1:1.5, about 1:0.1 to about 1:1, or about 1:1.

In some embodiments, a weight ratio of 5-HTP to GABA in the supplemental active ingredients may be about 1:0.1 to about 1:0.5, for example, about 1:0.1 to about 1:0.4, about 1:0.1 to about 1:0.3, or about 1:0.2. Similarly, a weight ratio of 5-HTP to melatonin in the supplemental active ingredients may be about 1:0.1 to about 1:0.5, for example, about 1:0.1 to about 1:0.4, about 1:0.1 to about 1:0.3, or about 1:0.2.

And in some embodiments, a weight ratio of GABA to melatonin in the supplemental active ingredients may be about 1:0.5 to about 1:2, for example, about 1:0.5 to about 1:1.5, about 1:0.1 to about 1:1, or about 1:1.

Methods of making the compositions according to embodiments of the present disclosure are not particularly limited, and any suitable methods may be used. In some embodiments, for example, compositions including the mixture of cannabinoids, the optional supplemental active ingredients and a carrier oil (e.g., to make a tincture) may be made by mixing the mixture of cannabinoids and supplemental active ingredients with the carrier oil until the mixture of cannabinoids and supplemental active ingredients are dissolved, dispersed or suspended in the carrier oil.

According to embodiments of the present disclosure, methods of making gummy compositions are also not particularly limited, and may be any suitable method, including conventional methods for making gummy compositions. For example, in some embodiments, the gelling component (e.g., pectin or gelatin) may be hydrated in the solvent (e.g., water), optionally in the presence of the buffer (e.g., sodium citrate). The sweetener (e.g., erythritol) and starch (e.g., tapioca syrup) may then be added, and the mixture mixed to combine all components. This mixture may be heated to remove some of the moisture and to promote gelation. The heating temperature is not particularly limited, and may be any suitable temperature. For example, in some embodiments, the mixture may be heated at a temperature of about 200° C. to about 250° C., for example about 220° C. to about 240° C. The gelation/coagulation promoter may then be added to the mixture to adjust the pH of the mixture and promote gelation. The flavorant(s) and colorant(s), when used, may be then be added to the mixture, and the mixture poured into a gummy mold to set up. It is understood, however, that the methods of making the gummy composition are not limited to this process, and the components of the gummy composition may be mixed/added in any order without limitation, and for example, may all be mixed together at once, rather than the step-wise process described here. Indeed, any known process for making a gummy composition may be used, and one example such process is disclosed in US Patent Publication No. 2020/0138705, titled “PECTIN GUMMY COMPOS[I]TION AND METHODS OF MAKING AND USING THEREOF,” to Wan et al., published May 7, 2020, the entire content of which is incorporated herein by reference.

In one example formulation, a tincture for promoting sleep may include 1000 mg CBD, 500 mg CBG, and 333 mg CBN dissolved or suspended in MCT oil to make 30 mL of tincture. In another example formulation, a gummy includes 10 mg CBD, 2 mg CBG, 2 mg CBN, 10 mg L-theanine, 10 mg 5-HTP, 2 mg GABA and 2 mg melatonin suspended or entrained in a pectin-based gummy having a weight of 6 g. The pectin-based gummy may be made from a composition including pectin, tapioca syrup, a sugar-free sweetener (i.e., erythritol), a colorant, a flavorant, citric acid, sodium citrate, and water. These formulations are provided here for illustrative purposes only, and do not limit the scope of the present disclosure.

And in another example formulation for the production of 550 gummies having a weight of about 6 g, the composition may include 5.52 g CBD, 1.15 g CBG, 1.15 g CBN, 5.5 g L-theanine, 5.5 g 5-HTP, 1.1 g GABA, 1.1 g melatonin, 120 g flavorant(s), 0.5 g to 20 g colorant(s), 10 g citric acid, 270 g combined pectin and sodium citrate mixture, 720 g water, 1257 g tapioca syrup, and 1532 g erythritol. Again, this formulation is provided here for illustrative purposes only, and does not limit the scope of the present disclosure.

According to some embodiments of the present disclosure, a method of promoting or improving sleep comprises administering the composition for promoting sleep disclosed herein to a user prior to a predetermined time for sleep. The administration may be by any suitable means, for example, by topical application to the skin or by oral administration. In some embodiments, for example, when the composition for promoting sleep includes a sleep tincture as described herein, the administration may include topical application of the tincture to skin prior to a dedicated or predetermined sleep time. Any suitable such topical administration may be used, e.g., rubbing the tincture into any suitable portion of skin, non-limiting examples of which may include rubbing the tincture on the temples, and/or any one or more pulse points (e.g., the neck (carotid artery), the wrist (radial artery), behind the knee (popliteal artery), the groin (femoral artery), inside the elbow (brachial artery), the foot (dorsalis pedis and posterior tibial artery), the abdomen (abdominal aorta)). Alternatively, the tincture may be administered orally, for example, by applying the tincture inside the mouth or on the tongue, by drinking the tincture, or by mixing the tincture with another liquid and drinking the liquid.

In some embodiments, for example, when the composition includes a gummy, the administration may be by oral administration. For example, the oral administration may be by ingesting the gummy or gummies. As described herein, the user may ingest 1 to 2 gummies prior to a dedicated or predetermined sleep time.

While certain exemplary embodiments of the present disclosure have been illustrated and described, those of ordinary skill in the art will recognize that various changes and modifications can be made to the described embodiments without departing from the spirit and scope of the present invention, and equivalents thereof, as defined in the claims that follow this description. For example, although certain components may have been described in the singular, i.e., “a” carrier, “an” active ingredient, and the like, one or more of these components in any combination can be used according to the present disclosure.

Also, although certain embodiments have been described as “comprising” or “including” the specified components, embodiments “consisting essentially of” or “consisting of” the listed components are also within the scope of this disclosure. For example, while embodiments of the present disclosure are described as comprising a dietary supplement or composition comprising a mixture of cannabinoids, a carrier, and optionally one or more supplemental active ingredients, embodiments consisting essentially of or consisting of these components are also within the scope of this disclosure. Accordingly, a dietary supplement or composition may consist essentially of a mixture of cannabinoids, a carrier, and optionally one or more supplemental active ingredients. In this context, “consisting essentially of” means that any additional components will not materially affect the chemical, physical, therapeutic, dietary or sleep promotive properties of the dietary supplement or composition.

As used herein, unless otherwise expressly specified, all numbers such as those expressing values, ranges, amounts or percentages may be read as if prefaced by the word “about,” even if the term does not expressly appear. Further, the word “about” is used as a term of approximation, and not as a term of degree, and reflects the penumbra of variation associated with measurement, significant figures, and interchangeability, all as understood by a person having ordinary skill in the art to which this disclosure pertains. Any numerical range recited herein is intended to include all sub-ranges subsumed therein. Plural encompasses singular and vice versa. For example, while the present disclosure may describe “a” carrier or “an” active ingredient, a mixture of such materials can be used. When ranges are given, any endpoints of those ranges and/or numbers within those ranges can be combined within the scope of the present disclosure. The terms “including” and like terms mean “including but not limited to,” unless specified to the contrary.

Notwithstanding that the numerical ranges and parameters set forth herein may be approximations, numerical values set forth in the example formulations are reported as precisely as is practical. Any numerical value, however, inherently contains certain errors necessarily resulting from the standard variation found in their respective testing measurements. The word “comprising” and variations thereof as used in this description and in the claims do not limit the disclosure to exclude any variants or additions.

What is claimed is:

1. A composition for promoting sleep, the composition comprising:
a mixture of cannabinoids comprising cannabidiol (CBD), cannabigerol (CBG), and cannabinol (CBN); and
a carrier.

2. The composition of claim 1, wherein:
the CBD is present in the mixture of cannabinoids in an amount of about 50 wt % to about 90 wt % based on a total weight of the mixture of cannabinoids;
the CBG is present in the mixture of cannabinoids in an amount of about 5 wt to about 30 wt % based on a total weight of the mixture of cannabinoids; and
the CBN is present in the mixture of cannabinoids in an amount of about 1 wt % to about 30 wt % based on a total weight of the mixture of cannabinoids.

3. The composition of claim 1, wherein a weight ratio of the CBG to the CBD in the mixture of cannabinoids is about 1:20 to about 1:1.

4. The composition of claim 1, wherein a weight ratio of the CBN to the CBD in the mixture of cannabinoids is about 1:50 to about 1:1.

5. The composition of claim 1, wherein a weight ratio of the CBN to about CBG in the mixture of cannabinoids is about 1:5 to about 1:1.

6. The composition of claim 3, wherein:
a weight ratio of the CBN to the CBD is about 1:50 to about 1:1, or
a weight ratio of the CBN to the CBG is about 1:5 to about 1:1.

7. The composition of claim 1, wherein the carrier comprises a carrier oil or a carrier matrix.

8. The composition of claim 7, wherein the carrier oil comprises a tree-based oil, a nut-based oil or a plant-based oil.

9. The composition of claim 7, wherein the carrier oil comprises a medium-chain triglyceride (MCT) oil, an olive oil, an avocado oil, an almond oil, or a hemp seed oil.

10. The composition of claim 7, wherein the carrier matrix comprises a gummy composition.

11. The composition of claim 10, wherein the gummy composition comprises a gelatin-based gummy base composition or a plant-based gummy base composition.

12. The composition of claim 1, further comprising one or more supplemental active ingredients.

13. The composition of claim 12, wherein the one or more supplemental active ingredients comprises one or more of L-theanine, 5-hydroxytryptophan (5-HTP), gamma aminobutyricacid (GABA), and melatonin.

14. The composition of claim 12, wherein a weight ratio of the mixture of cannabinoids to a sum total of the supplemental active ingredients is about 1:5 to about 1:2.

15. The composition of claim 12, wherein a sum total amount of the supplemental active ingredients in the composition is about 0.1 wt % to about 1 wt % based on a total amount of the mixture of cannabinoids, the supplemental active ingredients, and the carrier.

16. The composition of claim 12, wherein the one or more supplemental active ingredients includes each of L-theanine, GABA, 5-HTP and melatonin.

17. The composition of claim 13, wherein:
the L-theanine is present in an amount of about 20 wt % to about 100 wt % based on a total weight of the supplemental active ingredients; or
the 5-HTP is present in an amount of about 20 wt % to about 100 wt % based on a total weight of the supplemental active ingredients; or
the GABA is present in an amount of about 5 wt % to about 100 wt % based on a total weight of the supplemental active ingredients; or
the melatonin is present in an amount of about 5 wt % to about 100 wt % based on a total weight of the supplemental active ingredients.

18. The composition of claim 16, wherein:
a weight ratio of the L-theanine to the GABA in the supplemental active materials is about 1:0.1 to about 1:0.5; or
a weight ratio of the L-theanine to the 5-HTP in the supplemental active materials is about 1:0.5 to about 1:2; or
a weight ratio of the L-theanine to the melatonin in the supplemental active materials is about 1:0.1 to about 1:0.5; or
a weight ratio of the 5-HTP to the GABA in the supplemental active materials is about 1:0.1 to about 1:0.5; or
a weight ratio of the 5-HTP to the melatonin in the supplemental active materials is about 1:0.1 to about 1:0.5; or
a weight ratio of the GABA to the melatonin in the supplemental active materials is about 1:0.5 to about 1:2.

19. A method of promoting sleep, the method comprising administering to a user the composition of claim 1 prior to a predetermined sleep time.

20. The method of claim 19, wherein administering the composition comprises orally administering the composition.
